(19) United States
(12) Patent Application Publication
(10) Pub. No.: US 2026/0060944 A1
(43) Pub. Date: Mar. 5, 2026

(54) PHARMACEUTICAL COMPOSITION AND USE THEREOF

(71) Applicant: Kuei-Chung Shih, Taichung City (TW)

(72) Inventor: Kuei-Chung Shih, Taichung City (TW)

(21) Appl. No.: 19/107,043

(22) PCT Filed: Aug. 29, 2023
(86) PCT No.: PCT/CN2023/115614

§ 371 Date: Feb. 27, 2025

Related U.S. Application Data
(60) Provisional application No. 63/401,718, filed on Aug. 29, 2022.

(51) Int. Cl. A61K 31/167; A61K 31/64; A61K 33/06; A61K 33/26; A61K 33/30; A61K 33/32; A61K 33/34; A61P 35/00

(52) U.S. Cl. CPC A61K 31/167; A61K 31/64; A61K 33/06; A61K 33/26; A61K 33/30; A61K 33/32; A61K 33/34; A61P 35/00

(57) ABSTRACT

A pharmaceutical composition includes: (a) a targeting agent for cancer cell mitochondria, (b) an ion chelating agent, and (c) pharmacologically active multivalent ions. The weight ratio of (a) is 64.5%˜43.7% based on the total weight of the pharmaceutical composition, the weight ratio of (b) is 56.2%˜16.1% based on the total weight of the pharmaceutical composition; the weight ratio of (c) is 19.4%˜0.000116% based on the total weight of the pharmaceutical composition.

### BACKGROUND OF THE INVENTION

### 1. Field of the Invention

The present invention generally relates to a pharmaceutical composition, a kit containing the pharmaceutical composition, and its applications. Specifically, the present invention is directed to a pharmaceutical composition for treating malignant diseases of abnormal growth of animal cells, a kit containing the pharmaceutical composition, and the use of both the composition and the kit.

### 2. Description of the Prior Art

Cancer is a major global health challenge to affect not only humans but also other species. The development of malignant tumors results from genetic mutations leading to abnormal cells. These cells continuously divide and proliferate, forming tumors capable of invading and destroying surrounding normal tissues and organs.

Furthermore, they can metastasize to distant organs through the bloodstream or lymphatic system and may develop drug resistance, increasing the difficulty of treatment. The destroying malignant tumor cells exhibit aggressive behavior, high recurrence rates, and are hard to cure. The causes of cancer are very complex and there is still no complete cure. Therefore, it is crucial to develop drugs which effectively suppress cancer cell proliferation for improving patient outcomes.

Common cancer therapies include surgical interventions, radiation therapy, and chemotherapy, such as using target drugs for cancer therapies. Unfortunately, these conventional treatments, while inhibiting tumor growth, often have collateral damages or kill normal tissues. Alternative therapies designed to target and eliminate cancer cells without causing side effects are still under research and preclinical trials.

Currently, one of the most common and cost-effective targeted therapy drugs is Iressa. However, its annual treatment cost is approximately 540,000 NTD, even with partial reimbursement of Health Insurance under specific conditions, depending on hospital-specific pricing. This imposes a significant financial burden on patients. Thus, developing more affordable and effective drugs for cancer treatment which reduce the financial burden on patients is importance.

### SUMMARY OF THE INVENTION

In view of the foregoing, the present invention provides a pharmaceutical composition effective against malignant tumors, a kit containing the pharmaceutical composition, and their uses in treating malignant diseases of abnormal growth of animal cells. This pharmaceutical composition is applicable in the treating field of oncology. The pharmaceutical composition proposed by the present invention leverages functional interactions between various existing drugs to synergistically disrupt the growth mechanisms of cancer cells. The pharmaceutical composition proposed by the present invention is simple to produce, cost-effective, and demonstrates statistically significant efficacy in inhibiting the growth of malignant tumors. Additionally, the components of the composition may be administered separately and still exhibit inhibitory effects on cancer cell in individuals.

In one aspect, the present invention provides a pharmaceutical composition containing: (a) a mitochondrial targeting agent for cancer cells, (b) an ion chelating agent, and (c) pharmacologically active multivalent ions. The mitochondrial targeting agent for cancer cells is Niclosamide. The ion chelating agent is Disulfiram. The pharmacologically active multivalent ions are selected from a group consisting of magnesium ions, calcium ions, manganese ions, ferrous ions, copper ions, and zinc ions. The weight ratio of (a) is 64.5%˜43.7% based on the total weight of the pharmaceutical composition, a weight ratio of (b) is 56.2%˜16.1% based on the total weight of the pharmaceutical composition, and a weight ratio of (c) is 19.4%˜0.000116% based on the total weight of the pharmaceutical composition.

In one embodiment of the present invention, 0.091≤(a)/((b)+(c))≤15.548.

In another aspect, the present invention provides a use of the aforementioned pharmaceutical composition for preparing a medicament for treating malignant tumors in individuals in need.

In one embodiment of the present invention, the malignant tumors are selected from a group consisting of solid tumors and hematological malignant diseases.

In one embodiment of the present invention, the malignant tumors are selected from a group consisting of lung cancer and brain cancer.

In one embodiment of the present invention: (a) the dose per administration ranges from 2000 mg to 100 mg, (b) the dose per administration ranges from 500 mg to 128.6 mg, (c) the dose per administration ranges from 600 mg to 2.65×10⁻⁴ mg.

In one embodiment of the present invention, the administration of (c) is no later than the administration of (b).

In one embodiment of the present invention, the medicament is administered no more than twice per day.

In one embodiment of the present invention, the medicament is administered via an oral route, an injected route, a transdermal route, or an inhaled route.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 illustrates the correlation between the inhibition efficacy on tumor volume and administration time for the pharmaceutical composition of the present invention versus the conventional targeted drug against the human non-small cell lung cancer (A549) cell line in vivo.

FIG. 2 illustrates the correlation between inhibition efficacy on tumor weight and administration time for the pharmaceutical composition of the present invention versus the conventional targeted drug against the human non-small cell lung cancer (A549) cell line in vivo.

FIG. 3 illustrates the correlation between the inhibition efficacy on tumor volume and administration time for the pharmaceutical composition of the present invention versus the conventional targeted drug against the human lung squamous cell carcinoma (H520) cell line in vivo.

FIG. 4 illustrates the correlation between the inhibition efficacy on tumor weight and administration time for the pharmaceutical composition of the present invention versus the conventional targeted drug against the human lung squamous cell carcinoma (H520) cell line in vivo.

### DETAILED DESCRIPTION

Unless otherwise defined, all technical and scientific terms used herein have the same meanings as commonly understood by those of ordinary skilled in the art. The terminology used in this specification is solely for the purpose of describing particular embodiments and is not intended to limit the scope of the invention. Unless expressly stated otherwise, the terms used in the specification and the claims shall have the meanings set forth below.

Malignant tumor, neoplasm: also known as cancer, it refers to a disease of the abnormal growth and division of cells, characterized by cells which have the potential for unlimited proliferation, the ability to invade surrounding tissues and organs, and a tendency to metastasize to other parts of the body. The malignant tumors are complicated diseases involving multiple genetic mutations and molecular mechanisms. Early detection, precise diagnosis, and multidisciplinary treatment strategies are critical for controlling the progression of the malignant tumors. Treatment options include surgical excision, radiotherapy, chemotherapy, targeted therapy, and immunotherapy, depending on the patient's condition and the cancer type. The malignant tumors differ from benign tumors, which typically exhibit localized growth, minimal invasion of adjacent tissues, and rare metastasis to other sites.

Major characteristics of the malignant tumors include:
1. Unlimited Proliferation: the malignant tumor cells lose the regulatory mechanisms which control normal cell growth. They can continuously divide and proliferate to form tumor tissues.
2. Infiltration and Invasion: the malignant tumor cells can invade surrounding normal tissues and organs, and disrupt their structure and function. This invasiveness can make surgical excision challenging to make treatment complicated.
3. Metastasis: the malignant tumor cells can sometimes spread via the bloodstream or lymphatic system to other parts of the body, to form metastatic tumors (also called secondary tumors). This leads to the appearance of cancer in multiple locations to increase the treatment difficulty.
4. Abnormal Cell Morphology: the malignant tumor cells often exhibit morphological differences from normal cells, including irregular shapes, deformations, and size variability.
5. Heterogeneity: within the same malignant tumor, the cells may differ in their properties and characteristics, known as heterogeneity. This complicates treatment, as different subpopulations of cells may respond differently to the therapies.
6. Angiogenesis: the malignant tumors often induce the formation of new blood vessels to supply nutrients and oxygen, supporting their continuous growth.

Drug Synergy: also referred to as drug synergism, this occurs when the combined use of two or more drugs produces an effect greater than the sum of their individual effects. In drug therapy, drug synergy can result in stronger, more prolonged, or broader therapeutic outcomes while potentially reducing side effects associated with individual drugs. Drug synergy can be observed among various types of drugs, including antibiotics, anticancer drugs, and antiviral agents. However, not all drugs or diseases benefit from synergy, as interactions between drugs can be complex and variable. The concept of drug synergy can be further elaborated as follows:
1. Enhanced Efficacy: Drug synergy may lead to increased therapeutic effectiveness. This means that the combined effect of multiple drugs can be stronger to result in effectively controlling or treating diseases.
2. Reduced Resistance Risk: prolonged use of a single drug may result in the development of resistance in bacteria, viruses, or tumors. Using multiple drugs with different mechanisms may reduce the risk of resistance, maintaining the therapeutic efficacy.
3. Decreased Side Effects: in some cases, lower doses of multiple drugs can replace a high dose of a single drug, thereby reducing the risk of adverse side effects.
4. Multi-Targets Intervention: Different drugs may act on various targets or pathways of a disease, providing multiway interventions which enhance the diversity and comprehensiveness of treatment.
5. Addressing Complex Diseases: certain complex diseases involve multiple pathological mechanisms, making it difficult for a single drug to provide comprehensive intervention. Drug synergy can impact various aspects of a disease at multiple levels, effectively addressing complex conditions.

Hematologic Malignancies: these refer to abnormal proliferations of white blood cells and can be categorized as leukemia or lymphoma.

Cancer Cell death Rate: Refers to the percentage of cancer cells killed by a treatment (e.g., chemotherapy, radiotherapy, targeted therapy) within a given timeframe. It is an important method for evaluating the effectiveness of treatments and understanding their impact on cancer progression. The significance of the cancer cell death rate is as follows: Therapeutic Effectiveness: The cancer cell death rate is a critical indicator for evaluating the efficacy of cancer treatments. A higher death rate generally signifies a more effective method in killing cancer cells.

Disease Control: monitoring the death rate of cancer cells helps assess whether a treatment effectively controls or reduces the progression and spread of cancer. A low death rate may necessitate reevaluation of the treatment approach to achieve better control.

Prognosis Prediction: A higher cancer cell death rate may correlate with improved patient prognosis. Effectively killing cancer cells can lower the risk of disease recurrence and progression, thereby enhancing patient survival rates.

Treatment Selection: The death rate of cancer cells aids medical professionals in evaluating the advantages and drawbacks of various treatments. Treatment selection often considers death rates along with potential side effects, resistance, and other factors.

Therapy Monitoring: Tracking the death rate of cancer cells helps healthcare professionals monitor treatment progress. If the death rate does not show significant improvement over time, adjustments to the therapeutic regimen may be required.

The term “treat” includes partial or complete prevention, improvement, alleviation, and/or management of symptoms, secondary disorders, or conditions associated with cancer. As used in this specification, “treat” also refers to the administration or application of one or more pharmaceutical agents described in the present invention to an individual exhibiting symptoms, secondary disorders, or conditions related to cancer, aiming for partial or complete relief, alleviation, disease cure, delayed onset, suppression of disease progression, reduction in disease severity, and/or reduction in the incidence of one or more cancer-related symptoms, conditions, or secondary disorders. Cancer-related symptoms, secondary disorders, and/or conditions include, but are not limited to, fever, weakness, fatigue, weight loss, pain, coughing, bleeding, skin changes, diarrhea or constipation, nausea, vomiting, and loss of appetite. “Treatment” may also refer to administration to individuals with early symptoms or conditions to lower the risk of developing cancer-related symptoms, secondary disorders, and/or conditions. Here, “treatment” may effectively reduce one or more symptoms or clinical markers. In other words, treatment may involve reducing, delaying, or halting the progression of disease, symptoms, or conditions.

The term “effective amount” refers to an amount of a pharmaceutical agent sufficient to generate the intended therapeutic response. The term “effective amount” also refers to a compound or a composition which yields therapeutic benefits that outweigh its toxic or adverse effects. The specific effective amount depends on various factors, such as the condition to be treated, the physiological state of the patient (e.g., weight, age, or sex), the type of mammal or animal receiving the treatment, treatment duration, the current therapies (if applicable), and the formulation and chemical structure of the compound or its derivatives. For instance, an effective amount may be expressed as the total weight of the drug (e.g., in grams, milligrams, or micrograms) or as the drug-to-body-weight ratio (e.g., mg/kg). Alternatively, an effective amount may be expressed as the concentration of the active ingredient (the pharmaceutical agents of the present invention), such as in molar concentration, in weight concentration, in volume concentration, in weight molar concentration, in molar fraction, I weight fraction and in a mixing ratio. Specifically, the term “therapeutically effective amount” refers to a dosage of a pharmaceutical agent sufficient to alleviate or relief symptoms associated with cancer. A person of ordinary skill in the art may calculate human equivalent doses (HED) of a pharmaceutical agent (such as those described herein) based on animal model data. For instance, the maximum safe starting dose for humans can be estimated following FDA guidelines such as the “Estimating the Maximum Safe Starting Dose in Initial Clinical Trials for Therapeutics in Adult Healthy Volunteers.”

As used herein, the term “in vitro” refers to events occurring in an artificial environment, such as in test tubes, in reaction vessels, or in cell cultures, rather than within a living organism.

As used herein, the term “in vivo” refers to events occurring within a biological organism, such as in human cells or in non-human animal cells. For cell-based discriptions, this term may also denote events happening within living cells as opposed to external systems.

As used herein, the term “individual” refers to any living organism to which the provided vaccines are administered for experimental, diagnostic, preventive, cosmetic, and/or therapeutic purposes. Typical individuals include animals, such as mammals (e.g., mice, rats, rabbits, non-human primates, and/or humans). In certain embodiments, the individual is a human.

As used herein, the term “pharmaceutically acceptable” or “pharmacologically active” refers to substances that, within the bounds of reasonable medical judgment, can be safely administered to humans and animals for tissue contact without causing excessive toxicity, irritation, allergic reactions, or other complications.

As used herein, the term “effective amount” refers to an amount sufficient to prevent and/or delay the onset of symptoms when administered to an individual. It is understood by a person of ordinary skill in the art that an effective dose containing at least one unit dose is typically delivered through a dosage regimen.

While numerical ranges and parameters defining the broad scope of the present invention are approximate values, specific implementation examples are presented to show the related values as accurately as possible. Unless otherwise stated, numerical ranges described herein include their endpoints.

In the present invention, the terms “cycle” and “cycle of treatment or treatment cycle” are used interchangeably to refer to a period during which treatment is administered to a patient. Generally speaking, cancer treatments involve a rest period during which no treatment is administered to follow the completion of a treatment cycle. After this rest period, one or more additional treatment cycles may follow, and each is succeeded by another rest period.

Drug Repurposing/repositioning refers to a new therapeutic application of existing drugs that have been previously approved for other medical uses. This approach reduces the cost and time required for drug development while minimizing the risk of adverse drug reactions post-market.

In cancer treatment, the combination of drug repurposing with drug-combination strategies offers significant advantages over traditional novel-targeted drugs, primarily for the following reasons:

Repurposed drugs, through novel combination strategies, enable new applications for treating malignant tumors, to facilitate beneficial drug interactions.

Drug combinations can exploit synergistic effects among different drugs to disrupt cancer cell growth mechanisms.

Existing well-used clinical drugs with well-established data and relatively high safety profiles can be repurposed for new therapeutic uses.

Multiple molecular signaling pathways associated with cancer can be inhibited through distinct pharmacological mechanisms.

Treatment effectiveness can be enhanced while lowering the individual drug dosages to make the therapies safer.

Combination therapies of drugs for malignant tumors are significantly much more cost-effective than targeted drug therapies.

The cost of the medicament of the present invention for treating cancer is much lower than the out-of-pocket expenses of all current targeted drugs which are covered by the health insurance. For example, taking IRESSA (Gefitinib) as an example, the currently lowest-priced cancer targeted drug, patients still need to pay NT. 573 for a single drug and use it continuously every day even with the reimbursement of Taiwan's National Health Insurance. The price of a single medicament of the present invention used to treat cancer is only NT. 184. Although the cost of the medicament is not covered by the health insurance, the patients can significantly reduce the cost of treatment and the medical burden if it is possible in the future.

Surprisingly, according to various embodiments of the present invention, the inventor has developed a pharmaceutical composition that effectively fights off malignant tumors. The active components of this composition are known drugs that already have extensive clinical data and relatively high safety profiles. Compared to targeted therapies, drug repurposing strategy through a combination of drugs offers multiple significant advantages.

The present invention first proposes a pharmaceutical composition which includes: (a) a targeting agent for cancer cell mitochondria, (b) an ion chelating agent, and (c) pharmacologically active multivalent ions. These three agents form a medicament combination, and their proportions can be adjusted based on specific therapeutic requirements. The therapeutic doses of (a), (b), and (c) are in milligrams (mg).

The mitochondrial targeting agent for cancer cells may be Niclosamide, which targets mitochondria within cancer cells to induce cell cycle arrest, inhibit cell growth, and promote apoptosis. According to GlobalData, no finished proposal has been published for Niclosamide for cancer treatment as a standalone therapy. Based on the total weight of the pharmaceutical composition, the proportion of (a) ranges from 64.5% to 43.7%. Niclosamide's dosage regimen involves daily administration (up to two doses per day) over seven days. The effective dose of Niclosamide is 857.2 mg, with a therapeutic dose range of 100 mg to 2000 mg.

The ion chelating agent may be Disulfiram, which induces endoplasmic reticulum stress and autophagy to promote apoptosis, reduces angiogenesis, and serves as a radiosensitizer or ion chelating agent. Similar to Niclosamide, no company has developed Disulfiram for standalone cancer treatment. Based on the total weight of the pharmaceutical composition, the proportion of (b) ranges from 56.2% to 16.1%. The upper limit for a single dose of Disulfiram is 500 mg, while the lower limit is 128.6 mg. The effective dose of Disulfiram is 257.2 mg, with a therapeutic dose range of 128.6 mg to 500 mg.

The pharmacologically active multivalent ions refer to metal ions in pharmaceutically acceptable nutrient additive forms and their salts, such as chloride, oxide, hydroxide, gluconate, sulfate, glycinate, succinate, stearate, glycerophosphate, acetate, lactate, phosphate, borate, carbonate, fumarate, glutarate, fructoborate, cholate, malate, glycolate, lactate, oxalate, carbonate, tartrate, citrate, stearate, HAP chelates, HVP chelates, yeast chelates, but the present invention is not limited thereto. These polyvalent ions include divalent or more ions from typical or transition elements. Examples include at least one of magnesium, calcium, chromium, manganese, ferrous, copper, or zinc ions. Based on the total weight of the pharmaceutical composition, the proportion of (c) ranges from 19.4% to 0.000116%. The therapeutic dose range for these ions is from 1800 mg to 2.65×10⁻⁴ mg, with effective doses ranging from 0.139 mg to 5.3×10⁻³ mg. For instance, as an ion chelating agent, two molecules of Disulfiram can combine one pharmacologically active polyvalent ion to form an active pharmacological chelate. Table 1 shows the details of the dosage ranges for different pharmacologically active polyvalent ions.

TABLE 1
Dosage ranges for different pharmacologically
active polyvalent ions

| pharmacologically active | | | | | |
|---|---|---|---|---|---|
| polyvalent ions | upper dose | | effective dose | lower dose | |
| cupric ions | 8 | mg | 0.139 mg | 0.0695 | mg |
| zinc ions | 30 | mg | 0.139 mg | 0.0695 | mg |
| ferrous ions | 45 | mg | 0.113 mg | 0.0565 | mg |
| divalent manganese ions | 9 | mg | 0.121 mg | 0.0605 | mg |
| magnesium ions | 600 | mg | 0.053 mg | 0.0265 | mg |
| calcium ions | 1800 | mg | 0.088 mg | 0.044 | mg |

The components (a), (b), and (c) in the pharmaceutical composition of the present invention may optionally have different formulated dosage ratio ranges or combinations.

In one embodiment of the present invention, the dosage ratio of component (a) to component (b) may range from

$$0.2 ∼ 15.55 = \frac{( a )}{( b )} .$$

In another embodiment of the present invention, the dosage ratio of component (a) to component (c) may range from

$$0.166 ∼ 75.471 \times 1 {0}^{3} = \frac{( a )}{( c )} .$$

In another embodiment of the present invention, the dosage ratio of component (a) to component (b)+(c) may range from

$$0.091 ∼ 15.548 = \frac{( a )}{( b ) + ( c )} .$$

In another embodiment of the present invention, the dosage ratio of component (a)+(b) to component (c) may range from

$$0.381 ∼ 94.339 \times 1 {0}^{3} = \frac{( a ) + ( b )}{( c )} .$$

In another embodiment of the present invention, the dosage ratio of component (a)+(c) to component (b) may range from

$$0.2 ∼ 20.217 = \frac{( a ) + ( c )}{( b )} .$$

In another embodiment of the present invention, the dosage ratio of component (b) to component (a) may range from

$$0.064 ∼ 5 = \frac{( b )}{( a )} .$$

In another embodiment of the present invention, the dosage ratio of component (b) to component (c) may range from

$$0.214 ∼ 18.867 \times 1 {0}^{3} = \frac{( b )}{( c )} .$$

In another embodiment of the present invention, the dosage ratio of component (b) to component (a)+(c) may range from

$$0.049 ∼ 4.998 = \frac{( b )}{( a ) + ( c )} .$$

In another embodiment of the present invention, the dosage ratio of component (b)+(c) to component (a) may range from

$$0.064 ∼ 11 = \frac{( b ) + ( c )}{( a )} .$$

In another embodiment of the present invention, the dosage ratio of component (c) to component (a) may range from

$$13.35 \times 1 {0}^{- 6} ∼ 6 = \frac{( c )}{( a )} .$$

In another embodiment of the present invention, the dosage ratio of component (c) to component (b) may range from

$$5.3 \times 1 {0}^{- 5} ∼ 4.665 = \frac{( c )}{( b )} .$$

In another embodiment of the present invention, the dosage ratio of component (c) to component (a)+(b) may range from

$$1.019 \times 1 {0}^{- 5} ∼ 2.624 = \frac{( c )}{( a ) + ( b )} .$$

The pharmaceutical composition of the present invention, with the aforementioned dosage ratio ranges of components (a), (b), and (c), can achieve a corresponding cytotoxic effect on cancer cells.

In one embodiment of the present invention, when the ratio of

$$\frac{( a )}{( b ) + ( c )} = 3.87 ,$$

the cancer cell death rate observed in mouse experiments is 46%.

In another embodiment of the present invention, when the ratio of

$$\frac{( a )}{( b ) + ( c )} = 3.33 ,$$

the cancer cell death rate observed in mouse experiments is 50%.

In another embodiment of the present invention, when the ratio

$$\frac{( b ) + ( c )}{( a )} = 2.2 ,$$

the cancer cell death rate observed in mouse experiments is 44.3%.

In another embodiment of the present invention, when the ratio of

$$\frac{( b ) + ( c )}{( a )} = 3.3 ,$$

the cancer cell death rate observed in mouse experiments is 58%.

In another embodiment of the present invention, when the ratio of

$$\frac{( b ) + ( c )}{( a )} = 6.6 ,$$

the cancer cell death rate observed in mouse experiments is 86.5%.

The components in the pharmaceutical composition of the present invention may optionally have various drug administration sequences. According to one embodiment of the present invention, the first pharmaceutical may be (c) pharmacologically active multivalent ions, the second pharmaceutical may be (b) an ion chelating agent, and the third pharmaceutical may be (a) a targeting agent for cancer cell mitochondria. The administration sequence of the first pharmaceutical (c) pharmacologically active multivalent ions is not later than the administration of the second pharmaceutical (b) ion chelating agent to enhance ion concentration within the animal. There are no specific restrictions on the administration sequence of the third pharmaceutical (a) the targeting agent for cancer cell mitochondria.

In some embodiments of the present invention, the administration sequence of the first pharmaceutical (c), second pharmaceutical (b), and third pharmaceutical (a) may have the following embodiments:
1. (a) administered first, followed by (c), then (b).
2. (a)+(c) administered simultaneously, followed by (b).
3. (c) administered first, followed by (b), then (a).
4. (c)+(b) administered simultaneously, followed by (a).
5. (a), (c), and (b) administered simultaneously.

Optionally, the pharmaceutical composition of the present invention may also include one or more stabilizers, binders, fillers, disintegrants, excipients, or additives.

The pharmaceutical composition of the present invention may be formulated into a pharmaceutical kit for different administration sequences. For instance, the pharmaceutical composition of the present invention may be designed as a pharmaceutical kit for treating individuals diagnosed with or possibly having cancer. The pharmaceutical kit may include a first container, an optional second container, an optional third container, and, an optional fourth container.

The first container, the optional second container, and the optional third container may respectively contain at least one of the first, second, or third pharmaceuticals. According to one embodiment of the present invention, the first pharmaceutical may be (c) pharmacologically active polyvalent ions, the second pharmaceutical may be (b) ion chelating agent, and the third pharmaceutical may be (a) the mitochondrial targeting agents for cancer cells.

Another aspect of the present invention relates to a method for treating an individual diagnosed with or suspected of having cancer by using the pharmaceutical kit described herein. This method involves administering the pharmaceutical from the first container of the invention to the individual, administering the pharmaceutical from the optional second container to the individual, and administering the pharmaceutical from the optional third container to the individual.

The first container contains at least one of the first pharmaceutical, second pharmaceutical, and third pharmaceutical. Similarly, the optional second container contains at least one of the first pharmaceutical, the second pharmaceutical, and the third pharmaceutical. The optional third container contains at least one of the first pharmaceutical, the second pharmaceutical, and the third pharmaceutical. In one embodiment, the first pharmaceutical is (c) pharmacologically active polyvalent ions, the second pharmaceutical is (b) ion chelating agents, and the third pharmaceutical is (a) mitochondrial targeting agents for cancer cells.

The first effective dose range of the first pharmaceutical, (c) pharmacologically active polyvalent ions, is approximately 1800 mg to 2.65×10 4 mg. The second effective dose range of the second pharmaceutical, (b) ion chelating agent, is approximately 500 mg to 128.6 mg. The third effective dose range of the third pharmaceutical, (a) mitochondrial targeting agents, is approximately 2000 mg to 100 mg.

Various different materials such as glass, liposomes, or plastic may be used to prepare the first container, the second container, and the third container suitable for accommodating the pharmaceuticals described in the present invention. The first container may hold an effective dose of the pharmaceuticals or its pharmaceutical formulation. The second container may hold an effective dose of the pharmaceuticals or its pharmaceutical formulation. The third container may hold an effective dose of the pharmaceuticals or its pharmaceutical formulation. Additionally, the pharmaceutical kit may include user instructions accompanying the first container, the second container, and/or third container.

The pharmaceutical kit may optionally include a fourth container containing a pharmaceutically acceptable buffer, such as phosphate-buffered saline, a Ringer's solution, or a glucose solution. Other commercially or user-required materials may also be included, such as additional buffers, diluents, filters, needles, and syringes.

Another aspect of the present invention relates to a method of administering the pharmaceutical kit of the present invention to treat an individual in need (e.g., a cancer patient or an individual suspected of having cancer). This method includes: (1) administering the pharmaceutical from the first container of a first effective dose to an individual, (2) administering the pharmaceutical from the optional second container of a second effective dose to the individual, (3) administering the pharmaceutical from the optional third container of a third effective dose to the individual. The administration of the first container to the individual is no later than the administration of the second container to the individual. The administration of the third container to the individual is not earlier than the administration of the second container to the individual. The administration of the third container to the individual may be not earlier than the administration of the second container by 10 minutes. Pharmaceuticals in the pharmaceutical kit should not be administered more than twice a day.

At the first administration point, the pharmaceuticals in the first container are administered in the form of liposome, capsule, or tablet to the individual. At the second administration point, the pharmaceuticals are administered in the form of liposome, capsule, or tablet to the individual. At the third administration point, the pharmaceuticals are administered in the form of liposome, capsule, or tablet to the individual. According to some embodiments, for an individual of 60 kg human, it is administered to the individual of the first pharmaceutical: 10 mg to 4.42×10⁻⁶ mg per kilogram of body weight per day, the second pharmaceutical: 8.33 mg to 2.14 mg per kilogram of body weight per day, the third pharmaceutical: 33.33 mg to 1.67 mg per kilogram of body weight per day. According to a working embodiment of the present invention, administering 51.67 mg to 3.81 mg/kg/day of the pharmaceutical composition of the present invention effectively reduces cancer cell count in the individual, thereby demonstrating the anticancer efficacy of the pharmaceutical composition of the present invention.

In some embodiments of the present invention, the first container contains the third pharmaceuticals, the second container contains the first pharmaceuticals, and the third container contains the second pharmaceuticals. At the first administration point, the pharmaceuticals in the first container are administered to the individual; at the second administration point, the pharmaceuticals in the second container are administered to the individual; and at the third administration point, the pharmaceuticals in the third container are administered to the individual so that an administration sequence of the method of using the pharmaceutical kit of the present invention to treat an individual diagnosed with or suspected of having cancer is first (a), followed by (c), then (b).

In certain some embodiments of the present invention, the first container contains the first pharmaceutical, the second container contains the second pharmaceutical, and the third container contains the third pharmaceutical. At the first administration point, the pharmaceuticals in the first container are administered to the individual; at the second administration point, the pharmaceuticals in the second container are administered to the individual; and at the third administration point, the pharmaceuticals in the third container are administered to the individual, so that an administration sequence of the method of using the pharmaceutical kit of the present invention to treat an individual diagnosed with or suspected of having cancer is first (c), followed by (b), then (a).

In certain some embodiments of the present invention, the first container contains the first pharmaceuticals and the third pharmaceuticals, and the second container contains the second pharmaceuticals. At the first administration point, the pharmaceuticals in the first container are administered to the individual; at the second administration point, the pharmaceuticals in the second container are administered to the individual, so that an administration sequence of the method of using the pharmaceutical kit of the present invention to treat an individual diagnosed with or suspected of having cancer is first (a) and (c), followed by (b).

In certain some embodiments of the present invention, the first container contains the first pharmaceuticals and the second pharmaceuticals, and the second container contains the third pharmaceuticals. At the first administration point, the pharmaceuticals in the first container are administered to the individual; at the second administration point, the pharmaceuticals in the second container are administered to the individual, so that an administration sequence of the method of using the pharmaceutical kit of the present invention to treat an individual diagnosed with or suspected of having cancer is first (c) and (b), followed by (a).

In certain some embodiments of the present invention, the first container contains the first pharmaceuticals, the second pharmaceuticals, and the third pharmaceuticals. At the first administration point, the pharmaceuticals in the first container are administered to the individual, so that an administration sequence of the method of using the pharmaceutical kit of the present invention to treat an individual diagnosed with or suspected of having cancer is (a), (b), and (c).

Regarding the release form of the pharmaceutical composition of the present invention, it may be formulated into bilayer or trilayer tablets. The inner layers are formulated by using sustained-release excipients combined with the pharmaceuticals, resulting in a release profile where the outermost layer releases the drug most rapidly, while the innermost layers release it progressively slower. The outer layer including fast-disintegrating excipients that combine with the pharmaceuticals are formulated by with the inner layers into tablets. By leveraging differing disintegration rates, pharmacologically active multivalent ions can be rapidly absorbed into the body, increasing their systemic concentration before interacting with later-released ion chelating agents to chelate for actions.

### Excipient for Rapid-Release Dosage Forms

Rapid-release dosage forms aim to release the drug quickly to achieve a swift therapeutic effect. In some embodiments of the present invention, excipients for preparing fast-disintegrating formulations may include, but are not limited to:
1. Lactose: As a commonly used auxiliary excipient in oral tablets, lactose exhibits excellent compressibility and fast-disintegration properties, facilitating rapid drug release.
2. Corn Starch: A natural polysaccharide excipient known for its good compressibility and disintegration capabilities, ideal for preparing fast-disintegrating tablets and granules.
3. Microcrystalline Cellulose: Derived from cellulose, this fine crystalline excipient is commonly used to prepare oral tablets and enhances the mechanical strength of tablets while promoting rapid drug release.
4. Water-Soluble Polymers: Examples of some water-Soluble Polymers include hydroxypropyl methylcellulose (HPMC) and polyethylene glycol (PEG). They may be used as a disintegrant when preparing a rapidly disintegrating dosage. They interact with water quickly, enabling the tablet to disintegrate rapidly and release the drug.
5. Protein Excipients: Certain proteins, such as gelatin and sodium starch glycolate, swell and disintegrate rapidly under moist conditions, ensuring swift drug release. These excipients may be used in fast-disintegrating tablets, granules, and capsules.

### Excipients for Sustained-Release Formulations

Sustained-release formulations usually use special excipients. In some embodiments of the present invention, they extend the drug release duration, allowing the drug to enter the bloodstream or target tissues at a controlled rate. In some embodiments of the present invention, such excipients may include, but are not limited to:
1. Polymers: Polymers are common excipients, such as polyethylene glycol (PEG) and poly(lactic-co-glycolic acid) (PLGA). They may form drug carriers that enable controlled release.
2. Silicates: Silicate excipients, such as silica dioxide and aluminum hydroxide, may form drug-coating layers or network structures to delay drug release.
3. Polylactic-co-glycolic acid, PLGA Microspheres: These microspheres may be used for injectable or oral formulations to serve as a carrier for sustained-release dosage forms. The size and structure of the microspheres may be adjusted to control the drug release rate based on different needs.
4. Liposomes: Liposomes are tiny particles composed of a lipid bilayer to wrap drugs and form stable carriers. Liposomes may be taken orally, injected, or applied topically to achieve slow release of drugs.
5. Polymeric liposomes: Polymeric liposomes are an improved version of liposomes. By adding polymers or macromolecule substances, the release rate and stability of the drug can be further adjusted.

The release forms of the pharmaceutical compositions of the present invention are not limited to the aforementioned excipients and techniques for preparing sustained-release formulations. Suitable excipients may be selected based on factors such as the properties of the drugs, therapeutic requirements, and the routes of administration.

In some embodiments of the present invention, the effects of the drugs on cancer or tumor cells may vary depending on the influence of neutral, negatively charged, or positively charged liposomes. The interactions of differently charged liposomes with cells may be discussed in terms of two perspectives:
1. The interaction between liposomes and cells.
2. The impact of liposomes on the delivery of the drug to cells.

These differences and impacts are described as follows:

Mechanisms of Interaction: Positively charged liposomes exhibit electrostatic attraction to negatively charged cell membranes, making them more likely to bind to the membrane. Conversely, negatively charged liposomes are less likely. There is no significant electrostatic interactions for neutral liposomes. These interaction mechanisms determine the affinity and uptake efficiency of different liposomes with cells.

Cellular Uptake Efficiency: Positively charged liposomes, due to their interaction with negatively charged cell membranes, enhance cellular uptake in cells. Primarily the cellular uptake is enabled through endocytosis. Once bound to the membrane, the uptake of the positively charged liposomes into cells may be active or passive. In contrast, negatively charged liposomes experience lower uptake efficiency owing to their electrostatic repulsion.

Intracellular Delivery Efficiency: Positively charged liposomes enhance the release and intracellular delivery of loaded substances, such as drugs or genetic materials, because they can interact with negatively charged intracellular molecules like nucleic acids and proteins. While negatively charged liposomes can also deliver drug or loaded substances to some extent, they work better in stabilizing and protecting drugs rather than direct intracellular delivery compared with positively charged liposomes. There is no electrostatic interaction between the neutral liposomes and cell membranes, they may minimize nonspecific interactions with cells under some circumstances, reducing the possibilities of adsorption and endocytosis. This improves the in vivo stability and bioavailability of the liposomes. They generally have a higher drug-loading capacity to more effectively encapsulating and stabilizing various drugs (hydrophilic and lipophilic).

In some embodiments of the present invention, positively charged liposomes include, but are not limited to, DOTAP (1,2-dioleoyl-3-trimethylammonium-propane), DDAB (dimethyldioctadecylammonium), DPTAP (dipalmitoylphosphatidylcholine), DMTAP (dimyristoylphosphatidylcholine), DMTMA (dimyristoyltrimethylammonium), DC-Chol (3β-[N—(N′,N′-dimethylaminoethane)-carbamoyl]cholesterol), DOTMA (1,2-dioleoyloxy-3-(trimethylammonium) propane), DPTMA (dipalmitoyltrimethylammonium), DPTTA (dipalmitoyltriethylammonium), and DMRIE (dimyristoyl-N,N-dimethylphosphatidylethanolamine).

In certain embodiments of the invention, negatively charged liposomes include, but are not limited to, DOPC (dioleoylphosphatidylcholine), DOPS (dioleoylphosphatidylserine), DOPG (dioleoylphosphatidylglycerol), DOPA (dioleoylphosphatidic acid), DOPS-Na (sodium dioleoylphosphatidylserine), POPG (palmitoyloleoylphosphatidylglycerol), SOPC (stearoyloleoylphosphatidylcholine), SOPG (stearoyloleoylphosphatidylglycerol), POPE (palmitoyloleoylphosphatidylethanolamine), and POPA (palmitoyloleoylphosphatidic acid).

In certain embodiments of the invention, neutral liposomes include, but are not limited to, DSPC (distearoylphosphatidylcholine), DMPC (dimyristoylphosphatidylcholine), DPPC (dipalmitoylphosphatidylcholine), DOPC (dioleoylphosphatidylcholine), DPPG (dipalmitoylphosphatidylglycerol), DOPG (dioleoylphosphatidylglycerol), DPPA (dipalmitoylphosphatidic acid), DOPA (dioleoylphosphatidic acid), DMPE (dimyristoylphosphatidylethanolamine), and DMPS (dimyristoylphosphatidylserine).

In some embodiments of the present invention, other components for the liposome preparation include, but are not limited to, cholesterol, polyethylene glycol (PEG), surfactants (e.g., Tween, Span), excipients (e.g., phosphatidylserine, phosphatidic acid), liposome encapsulation agents (e.g., phosphatidylcholine), targeting ligands (e.g., antibodies, ligands), dyes (e.g., fluorescent dyes, nucleic acid dyes), and liposome stabilizers (e.g., liposome structural modulators, preservatives).

In some embodiments of the present invention, drugs can be encapsulated using small and large capsules. For example, two drugs—an ion chelating agent and a mitochondrial targeting agent for cancer cells—can be first encapsulated within small capsule liposomes. These small capsule liposomes are then further encapsulated along with pharmacologically active multivalent ions within large capsule liposomes, forming a two-layered liposomal structure. Upon administration, the large liposomes disintegrate first, releasing the multivalent active ions to increase local concentrations, then the small liposomes disintegrate, releasing the ion chelating agent and the mitochondrial targeting agent. The ion chelating agent then interacts with the previously released active multivalent ions, forming a chelate that exerts therapeutic effects. For instance, the large liposomes may disintegrate, followed by the disintegration of the small liposomes within 10 minutes.

In some embodiments of the present invention, a dual-layer or triple-layer liposome is a process used to encapsulate and deliver three distinct drugs simultaneously to have a multilayered liposomal structure, for example to enhance the stability of individual drugs by encapsulating each drug in its liposome layer, protecting them from degradation, chemical interactions, and enzymatic breakdown, or for controlled drug release, as the structure of dual-layer liposomes enables individual release regulation for each drug. By adjusting the composition and structure of the liposomes, the independent release of each drug is enabled, thereby precisely controlling the release rate and timing of each drug. This helps maintain the drug stability and integrity during storage and transport. This approach enhances the effectiveness of drug delivery strategies, to increase drug stability and to reduce the likelihood of drug degradation or inactivation and improving biological efficacy. Therefore, by adjusting the lipid components of the inner and outer layers of the double-layer or triple-layer liposomes, it can be beneficial to control and regulate the drug delivery process, such as disintegrating the drug before pharmacologically multivalent active ions enter the body in advance to increase the body concentration and then chelate with the released ion chelating agent. Therefore, using liposomes of different sizes for coating is a strategy that can achieve the gradual release of drugs. By first coating one of the drugs with small liposomes, and then using large particle size liposomes to coat pharmacologically multivalent active ions and small particle size liposomes. During the release process, the large liposomes can disintegrate first, allowing the pharmacologically multivalent active ions therein to play a role in increasing the concentration in the body, while the small liposomes later disintegrate and release the ion chelating agent to produce a chelation reaction, delaying the release of the drug to optimize the therapeutic effect and reduce drug side effects.

Example cancer indications of the pharmaceutical kit and/or composition described in the present invention include malignant tumors. The malignant tumors include, but are not limited to, solid tumors or hematological malignant diseases, such as gastric cancer, lung cancer, bladder cancer, breast cancer, pancreatic cancer, renal cancer, colorectal cancer, cervical cancer, ovarian cancer, brain cancer, prostate cancer, liver cancer, melanoma, esophageal cancer, multiple myeloma, and head and neck squamous cell carcinoma. According to some embodiments of the present invention, the cancer cells of the cancer exhibit resistance to at least one form of therapy, such as chemotherapy, radiotherapy, or immunotherapy. For example, there is few way to treat triple-negative breast cancer (TNBC), characterized by the lack of estrogen receptor (ER), progesterone receptor (PR), and HER2 expression, because it is not susceptible to hormonal and targeted therapies. Similarly, the drug-resistant human breast cancer cell line MCF7-R exhibits comparable resistance issues. Furthermore, glioblastoma cell lines like U-87 are known for high resistance to apoptosis and to targeted therapies such as IRESSA (Gefitinib) and ABT-737 (Bcl-2 inhibitor) (Chang et al., 2011; Cristofanon and Fulda, 2012; Jane et al., 2013). Additionally, human lung squamous carcinoma (H520) tumors display resistance to IRESSA, leading to inferior results of targeted treatments.

The examples of the administration methods for the pharmaceutical kit and/or composition of the present invention include, but are not limited to, enteral, oral, nasal, non-oral, topical, or mucosal routes. Non-oral routes may include intratumoral, intramuscular, intravenous, intra-arterial, subcutaneous, intraperitoneal, intracranial, intraventricular, or intrathecal injections.

### Experimental Procedures

### Materials and Methods for Animal Studies

### Experimental Animals

Female, 5-week-old Balb/c CAnN.Cg-Foxnlnu/CrlNarl nude mice were obtained from the National Laboratory Animal Center. They were provided with ad libitum access to water and feed (LabDiet 5058, PMI Nutrition International Inc., MO, USA; 21.56% energy from fat, metabolizable energy 3.46 kcal/gm). Environmental conditions were maintained at 22±2° C., with 55±15% humidity and a 12-hour light/dark cycle (lights on at 1 a.m., lights off at 1 p.m.). Experiments commenced when they reached six weeks of age.

### Cancer Induction in Animal Models

Nude mice of six weeks of age were used to establish two lung epithelial carcinoma and animal models. 1×10⁷ cells of Human non-small cell lung cancer (NSCLC) cell line A549 and of human lung squamous carcinoma cell line H520, provided by the Food Industry Research and Development Institute in Taiwan, were subcutaneously injected 100 μL into the dorsal area near the right hind limb. Tumor diameters were monitored, and reached 5-6 mm after two weeks, the mice were grouped for drug administration, successfully establishing the models.

### Drug Administration

The mice with established A549 or H520 tumors were divided into six groups for each tumor type.
S1: Control group (no treatment),
S2: Single group (Niclosamide),
S3: Single group (Disulfiram+multivalent ions),
S4: Targeted therapy group (IRESSA, Gefitinib),
S5: Effective dose combination group (Niclosamide+Disulfiram+multivalent ions),
S6: Low-dose combination group (Niclosamide+Disulfiram+multivalent ions).

A549 model: 6 mice per group, administered orally once daily for 25 days.

H520 model: 7 mice per group, administered orally once daily for 13 days. The pharmacologically active multivalent ions used were copper ions in the form of copper gluconate.

### Observations on Body Weight and Food Intake

During the experimental period, nude mice were allowed ad libitum access to food and water. Weekly body weight and food consumption were recorded using an electronic balance.

### Tumor Volume, Weight, and Inhibition Rate Assessment

Tumor volume was measured weekly using a tumor measurement device (TM900, Peira, Turnhout, Belgium). After five weeks of drug administration, the mice were sacrificed, and tumors were excised from the right hind limb. Tumor volume was calculated based on the longest and shortest diameters using the formula:

$$Volume = 0.5 \times Longest Diameter \times {( Shortest Diameter )}^{2} .$$

The tumors were then weighed and recorded. Tumor inhibition rates were calculated using the control group as a baseline:

$$Tumor Volume Inhibition Rate ( \% ) = 1 - ( Tumor Volume in Drug Group ) / ( Tumor Volume in Control Group ) \times 100 \%$$

$$Tumor Weight Inhibition Rate ( \% ) = 1 - ( Tumor Weight in Drug Group ) / ( Tumor Weight in Control Group ) \times 100 \%$$

### Materials and Methods for Cancer Cell Experiments

### Cell Culture

Human cancer cell lines, including non-small cell lung cancer (A549), glioblastoma (U87), glioblastoma (T98G), erythroleukemia type cell line (K-562), acute monocytic leukemia (THP-1), hepatocellular carcinoma (HepG2), colorectal cancer (HCT116), ovarian cancer (TOV-21G), triple-negative breast cancer (MDA-MB-231), or drug-resistant human breast cancer (MCF7-resistant), were cultured in media supplemented with 10% (v/v) fetal bovine serum (FBS) (Penicillin-Streptomycin Solution, ACE Biolabs, CC1009), glutamine-enriched Nutrient Mixture F-12 Ham Kaighn's Modification (F-12/K, SIGMA-ALDRICH, USA), and RPMI-1640 (HiMedia Laboratories LLC, USA). The cell lines were separately incubated in a 5% CO2 atmosphere at 37° C. using a controlled incubator (Astec-SCA-165DS).

### Cell Viability

Different cancer cell lines were seeded in 96-well plates and allowed to adhere for 24 hours. Cells were then treated with combinations of three drugs, while untreated cells served as the control group. 20 μL of MTS reagent (Promega, USA) was added to each well and incubated at 37° C. for one hour. Optical density (OD) was measured at 490 nm using an ELISA detector (BioTek EPOCH2) to evaluate the ell viability and proliferation by using the following formula:

$$Cell Viability ( \% ) = ( OD of Treated Cells ) / ( OD of Untreated Cells ) \times 100 \%$$

### Drug Dosage for Cancer Cell Lines Assay

In some embodiments of the present invention, drug concentrations used for testing cancer cell lines is in nM and the effective dose range ranged from 0.8 nM to 83 nM.

### Example 1: Efficacy of Different Pharmacologically Active Ions in the Pharmaceutical Composition of the Present Invention Tested on A549 Cells

To evaluate whether the pharmaceutical composition containing various pharmacologically active ions could produce synergistic drug effects, tests were conducted on human non-small cell lung cancer (A549) cell lines. The efficacy of inhibiting cancer cell survival rate was analyzed, and the results are presented in Table 2. The dose units in Table 2 are expressed in nM. The pharmacologically active ion used in this study was gluconate ion.

TABLE 2
Cell Survival Rate of A549 Cells Treated with the Pharmaceutical
Composition Containing Different Pharmacologically Active
Multivalent Ions of the Present Invention

| | | | |
|---|---|---|---|
| | | Pharmacologically | Survival |
| Niclosamide | Disulfiram | Active Ions | Rate (%) |

| | | | | |
|---|---|---|---|---|
| 6 | — | — | — | 98.00 |
| — | 1 | — | — | 99.58 |
| — | — | 1 | zinc | 104.71 |
| — | — | 1 | manganese | 104.35 |
| — | — | 1 | magnesium | 100.52 |
| — | — | 1 | ferrous | 106.27 |
| — | — | 1 | cupric | 103.70 |
| — | — | 1 | calcium | 103.98 |
| 6 | 1 | 1 | zinc | 51.97 |
| 6 | 1 | 1 | manganese | 52.75 |
| 6 | 1 | 1 | magnesium | 43.84 |
| 6 | 1 | 1 | ferrous | 41.73 |
| 6 | 1 | 1 | cupric | 21.32 |
| 6 | 1 | 1 | calcium | 49.60 |

The “-” symbol in the tables indicates that the specific component was not present in the composition.

The experimental results in Table 2 demonstrate that Niclosamide, Disulfiram, and pharmacologically active multivalent ions did not individually exhibit significant inhibitory effects on cancer cell lines. However, when different combinations of ions were incorporated into the pharmaceutical composition of the present invention, a positive inhibitory effect on cancer cells was observed. Based on experimental results, the composition containing pharmacologically active copper ions yielded the most effective inhibitory outcome.

The experimental results in Table 2 further indicate that while individual components of the pharmaceutical composition of the present invention did not significantly suppress the survival rate of human non-small cell lung cancer cells (A549), the use of different ion combinations in the present invention significantly reduced the survival rate of these cancer cells.

### Example 2: Efficacy of the Pharmaceutical Composition of the Present Invention on A549 Cells

To evaluate whether varying dosages of the pharmaceutical composition of the present invention could inhibit the survival rate of human non-small cell lung cancer cell line (A549), cell survival analyses of A549 were conducted. The results are shown in Table 3.

As illustrated in Table 3, the survival rate of A549 cells is dose-dependent when treated with the pharmaceutical composition of the present invention. In this experiment, the pharmacologically active copper ion was cupric in the form of copper gluconate.

TABLE 3
Effects of the Pharmaceutical Composition of the Present
Invention on the Survival Rate of A549 Cells

| Niclosamide | Disulfiram | Cupric | Survival Rate (%) |
|---|---|---|---|
| 2 | — | — | 112.66 |
| — | 80 | — | 93.85 |
| — | — | 80 | 109.28 |
| — | 25 | 12.5 | 64.42 |
| — | 25 | 25 | 47.63 |
| 1 | 25 | 25 | 21.80 |
| 2 | 12.5 | 12.5 | 70.76 |
| 4 | 12.5 | 12.5 | 41.96 |
| 4 | 25 | 25 | 13.46 |
| 6 | 0.8 | 0.8 | 43.89 |
| 6 | 1 | 1 | 37.05 |
| 13 | 0.8 | 0.8 | 39.88 |
| 13 | 1 | 1 | 33.70 |

### Observations from Table 3

The results in Table 3 indicate that Niclosamide, Disulfiram, and pharmacologically active copper ions did not individually exhibit significant inhibitory effects on cancer cell lines. However, when combined in various dosages the pharmaceutical composition of the present invention, effective inhibition was observed. When the concentration of Niclosamide was higher than that of Disulfiram and copper ions, or the concentration of Disulfiram and copper ions is higher than that of Niclosamide, better inhibitory effects on cancer cells are observed. This suggests a synergistic interaction among the drugs in the composition.

The experiment results show that no significant reduction in survival rate is observed when Niclosamide, Disulfiram, and copper ions were individually administered to A549 cancer cell lines at concentrations ranging from 2 to 80 nM. However, when Disulfiram and copper ions were co-administered at a concentration of 25 nM each, the survival rate decreased to 47.63%. Furthermore, when the pharmaceutical composition of the present invention were administered, the survival rate of A549 cells dropped significantly, reaching as low as 13.46%.

These experiment results suggest that as far as the pharmaceutical composition of the present invention is concerned a higher degree of synergistic efficacy is achieved when all three pharmaceuticals are used in combination.

The experimental results demonstrate that the pharmaceutical composition of the present invention effectively inhibits the survival rate of lung epithelial carcinoma cells. This inhibitory effect correlates positively with the dosage proportions of each pharmaceutical in the pharmaceutical composition of the present invention.

### Example 3: In Vivo Tumor Suppression Efficacy on A549 Cells of the Pharmaceutical Composition of the Present Invention

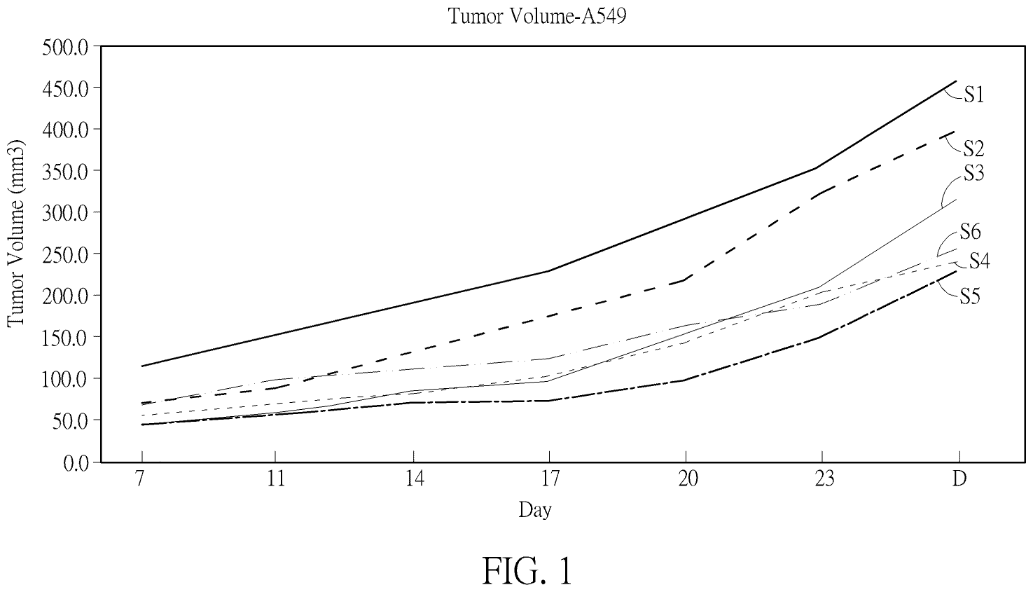

To evaluate whether the pharmaceutical composition of the present invention effectively inhibits non-small cell lung cancer, whether oral absorption via the gastrointestinal tract suppresses lung cancer tumor growth and whether adverse effects are present, in vivo experiments were conducted. A tumor model was established in nude mice via subcutaneous injection. Tumor size was monitored for 14 days, after which the mice were divided into six groups and treated with the pharmaceutical composition. The pharmaceutical-using group continued to be fed the test drug by oral gavage. Tumor growth in the nude mice of the tumor model was evaluated over 25 days of treatment, with tumor suppression effects of the tested pharmaceuticals on tumor shown in Tables 4 and 5. FIG. 1 illustrates the correlation between the inhibition efficacy on tumor volume and administration time for the pharmaceutical composition of the present invention versus the conventional targeted drug against the human non-small cell lung cancer (A549) cell line in vivo.

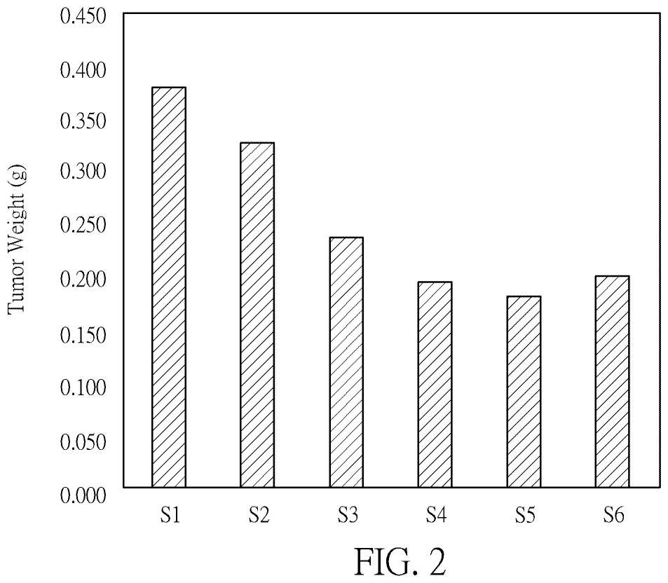

FIG. 2 illustrates the correlation between inhibition efficacy on tumor weight and administration time for the pharmaceutical composition of the present invention versus the conventional targeted drug against the human non-small cell lung cancer (A549) cell line in vivo.

TABLE 4
Correlation Between the Pharmaceutical Composition of the Present
Invention and the Conventional Target Drug Group on the Tumor
Volume of (A549) Cells and the Date of Administration

| | Day | | | | | | when |
|---|---|---|---|---|---|---|---|
| | 7 | 11 | 14 | 17 | 20 | 23 | sacrificed |
| S1 | 113.4 | 153.4 | 189.1 | 231.2 | 293.1 | 354.8 | 454.1 |
| S2 | 73.4 | 89.7 | 136.3 | 179.6 | 219.0 | 319.8 | 396.1 |
| S3 | 44.9 | 59.1 | 83.3 | 98.6 | 151.4 | 210.6 | 316.1 |
| S4 | 54.2 | 69.5 | 82.0 | 102.8 | 141.3 | 202.6 | 239.6 |
| S5 | 44.2 | 55.5 | 70.6 | 73.6 | 98.4 | 150.9 | 229.3 |
| S6 | 69.9 | 97.9 | 106.7 | 123.8 | 162.4 | 189.8 | 254.1 |

The tumor volumes in Table 4 are expressed in mm³.

In Table 4, the term “Day” represents the date of administration.

Table 4 shows the tumor volume inhibition rates in mice implanted with human non-small cell lung cancer cells. At the time of sacrifice, tumor volume inhibition rates compared to Group S1 are:
Group S2: 12.78%,
Group S3: 30.39%,
Group S4: 47.23%,
Group S5 (effective dosage group of the pharmaceutical composition): 49.51%,
Group S6 (low-dosage group of the pharmaceutical composition): 44.03%. Accordingly, these experiment results suggest that Group S5 exhibits more effective tumor inhibition compared to Group S4. Furthermore, Group S6 also outperforms Groups S2 and S3, confirming the superior tumor volume inhibition efficacy of the pharmaceutical composition of the present invention.

TABLE 5
Correlation Between the Pharmaceutical Composition of the
Present Invention and the Conventional Target Drug Group
on the Tumor Weight of (A549) and the Administered Drug
Dosages. The tumor weight is measured in grams (g).

| S1 | S2 | S3 | S4 | S5 | S6 |
|---|---|---|---|---|---|
| 0.381 | 0.330 | 0.239 | 0.197 | 0.184 | 0.20 |

Table 5 shows the tumor weight inhibition rates in mice implanted with human non-small cell lung cancer cells. Following sacrifice, the average tumor weight inhibition rates compared to Group S1:
Group S2: 13.5%,
Group S3: 37.3%,
Group S4: 48.3%,
Group S5 (effective dosage group of the pharmaceutical composition): 51.8%,
Group S6 (low-dosage group of the pharmaceutical composition): 47.4%.

Accordingly, the experiment results suggest that Group S5 exhibits more effective tumor weight inhibition compared to Group S4. Additionally, Group S6 outperforms Groups S2 and S3, confirming the superior tumor weight inhibition efficacy of the pharmaceutical composition of the invention.

Based on experimental results, comparison with the current lung cancer target drug it is evident that the pharmaceutical composition of the present invention effectively inhibits both tumor volume and tumor weight in vivo. The pharmaceutical composition of the present invention is efficiently absorbed through the gastrointestinal tract, enabling precise targeting of tumor tissues without adverse effects in animals during the administration of the pharmaceuticals.

### Example 4: In Vivo Tumor Suppression Efficacy on Human Lung Squamous Cell Carcinoma (H520) of the Pharmaceutical Composition of the Present Invention

To evaluate whether the pharmaceutical composition of the present invention has an inhibitory effect on human lung squamous cell carcinoma (H520), whether oral absorption via the gastrointestinal tract suppresses lung cancer tumor growth and whether without adverse effects are present, in vivo experiments were conducted. A tumor model was established in nude mice via subcutaneous injection. Tumor size was measured over 14 days, after which the mice were then divided into six groups and treated with the pharmaceutical composition. The pharmaceutical-using group continued to be fed the test drug by oral gavage. Due to the rapid growth rate of H520 tumors in vivo, animals were sacrificed when tumors reached a specific size. Tumor model nude mice were sacrificed after 13 days of administration to evaluate changes in tumor growth and observe the inhibitory effect of the test drug on tumors. Results are shown in Tables 6 and 7.

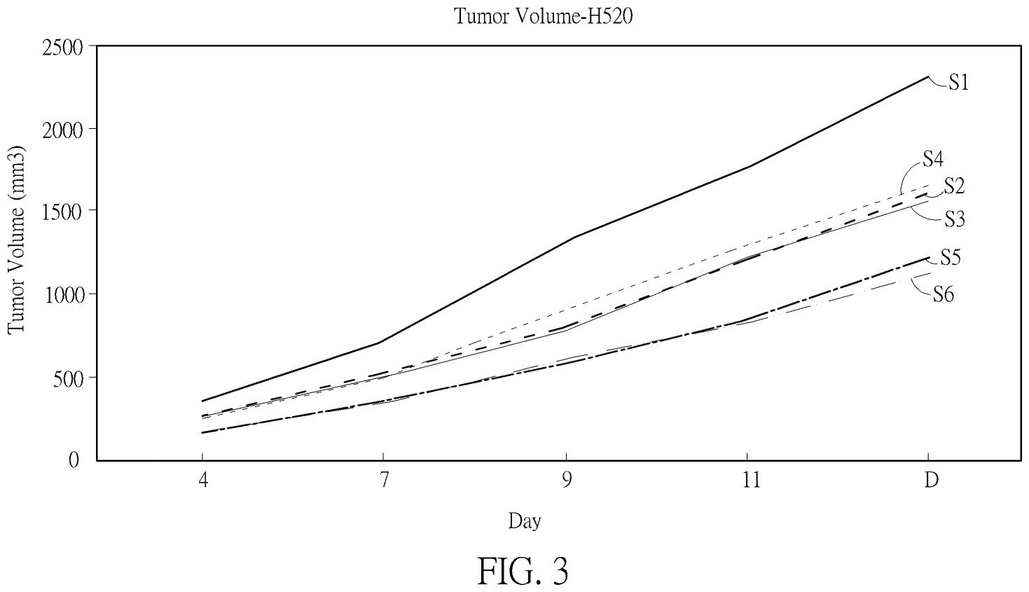

FIG. 3 illustrates the correlation between the inhibition efficacy on tumor volume and administration time for the pharmaceutical composition of the present invention versus the conventional targeted drug against the human lung squamous cell carcinoma (H520) cell line in vivo.

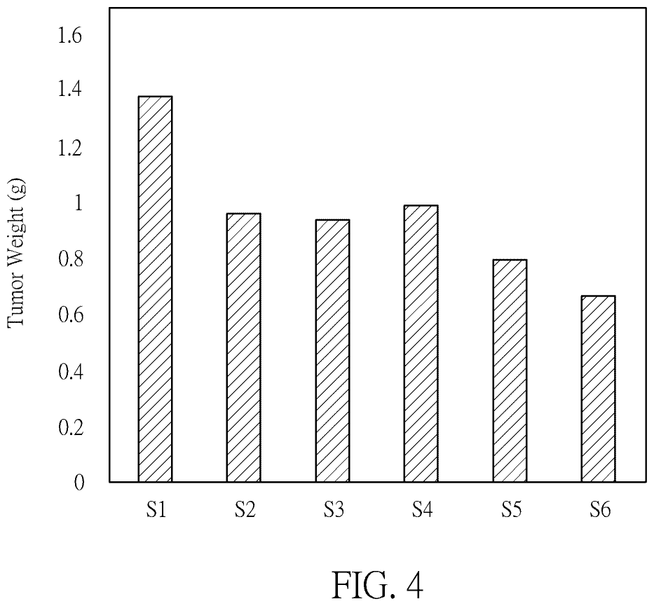

FIG. 4 illustrates the correlation between the inhibition efficacy on tumor weight and administration time for the pharmaceutical composition of the present invention versus the conventional targeted drug against the human lung squamous cell carcinoma (H520) cell line in vivo.

TABLE 6
Correlation Between the Pharmaceutical Composition of
the Present Invention and the Conventional Target Drug
Group on the Tumor Volume of (H520) Cells and the
Date of Administration. Tumor volume is measured in mm³.

| Day | 4 | 7 | 9 | 11 | when sacrificed |
|---|---|---|---|---|---|
| S1 | 366.4 | 721.4 | 1315.6 | 1759.9 | 2308.1 |
| S2 | 269.6 | 538.8 | 802.4 | 1204.1 | 1604.3 |
| S3 | 266.4 | 492.2 | 790.3 | 1216.3 | 1567.5 |
| S4 | 259.6 | 506.4 | 898.6 | 1290.0 | 1653.1 |
| S5 | 168.0 | 363.1 | 584.5 | 854.1 | 1216.3 |
| S6 | 171.7 | 338.4 | 613.3 | 828.7 | 1116.6 |

In Table 6, the term “Day” represents the date of administration.

Table 6 shows tumor volume inhibition rates in mice implanted with human lung squamous cell carcinoma. At the time of sacrifice, the tumor volume inhibition rates compared to Group S1 are:
Group S2: 30.49%,
Group S3: 32.09%,
Group S4 (targeted drug group): 28.38%,
Group S5 (effective dosage group of the pharmaceutical composition): 47.30%,
Group S6 (low-dosage group of the pharmaceutical composition): 51.62%. The results show that H520 cells exhibit higher resistance to the targeted drug in Group S4, resulting in the lowest tumor inhibition rate. Groups S2 and S3 shows lower inhibition rates than the pharmaceutical composition groups of the present invention to prove that the pharmaceutical composition of the present invention has a better effect on inhibiting the tumor volume of cancer cells. It is surprisingly found that the low-dosage Group S6 exhibits best tumor inhibition effect on this lung cancer type.

TABLE 7
Correlation Between the Pharmaceutical Composition of
the Present Invention and the Conventional Target Drug
Group on the Tumor Weight of (H520) and Administered
Drug Dosage. Tumor weight is measured in grams (g).

| S1 | S2 | S3 | S4 | S5 | S6 |
|---|---|---|---|---|---|
| 1.374 | 0.955 | 0.933 | 0.981 | 0.787 | 0.659 |

Table 7 shows the tumor weight inhibition rates in mice implanted human lung squamous cell carcinoma. The average tumor weight inhibition rates compared to Group S1 are: Group S2: 30.5%, Group S3: 32.1%, Group S4: 28.6%, Group S5 (effective dosage group of the pharmaceutical composition): 42.7%, Group S6 (low-dosage group of the pharmaceutical composition): 52.0%. Experimental analysis indicates that this tumor type exhibits higher resistance to the targeted drug in Group S4, owing to lower tumor inhibition rates in both volume and weight analyses, with the low-dose Group S6 showing superior results.

The experimental results show that the targeted drugs demonstrate inferior efficacy in treating human lung squamous cell carcinoma owing to tumor resistance. However, the pharmaceutical composition of the present invention effectively inhibits tumor growth for this cancer type without inducing resistance. It is surprisingly found that the low-dose Group S6 exhibits the best inhibition effect on this lung cancer tumor model.

The analysis of Examples 3 and 4 clearly shows that different lung cancer tumor types can develop resistance to targeted drugs, reducing therapeutic efficacy. The pharmaceutical composition of the present invention demonstrates significant inhibitory effects on various lung cancer tumor types without resistance issues.

### Example 5: Efficacy of the Pharmaceutical Composition of the Present Invention on Glioblastoma (T98G) Cell Line

To evaluate the inhibitory effect of the pharmaceutical composition of the present invention on the survival rate of glioblastoma (T98G) cell line, analysis on survival rate was performed. The results are shown in Table 8.

TABLE 8
The Effect of the Pharmaceutical Composition of the Present
Invention on the Survival Rate of Glioblastoma (T98G) Cell
Line. This cancer cell line is used in brain cancer research.

| Niclosamide | Disulfiram | Cu | Survival Rate (%) |
|---|---|---|---|
| 8 | — | — | 92.50 |
| 16 | — | — | 87.87 |
| — | 16 | — | 96.37 |
| — | 41 | — | 97.27 |
| — | 83 | — | 96.15 |
| — | — | 16 | 102.03 |
| — | — | 41 | 102.19 |
| — | — | 83 | 98.35 |
| 4 | 8 | 8 | 59.99 |
| 4 | 21 | 21 | 63.68 |
| 4 | 41 | 41 | 33.98 |
| 4 | 83 | 83 | 25.49 |
| 8 | 8 | 8 | 55.14 |
| 8 | 21 | 21 | 55.95 |
| 8 | 41 | 41 | 45.55 |
| 8 | 83 | 83 | 24.00 |
| 16 | 8 | 8 | 46.20 |
| 16 | 21 | 21 | 46.15 |
| 16 | 41 | 41 | 31.63 |
| 16 | 83 | 83 | 23.17 |

T98G is a human glioblastoma cell line commonly used in brain cancer research and drug development. As shown in Table 8, when Niclosamide, Disulfiram, and pharmacologically active multivalent Cu ions were individually tested, the results of this experiment show no significant inhibitory effects. Various concentrations of the pharmaceutical composition of the present invention demonstrates inhibitory effects. When the concentration of Niclosamide is greater than that of Disulfiram and Cu ions, or the concentration of Disulfiram and Cu ions is greater than that of Niclosamide, better inhibitory effects on cancer cells are observed. This suggests a synergistic interaction among the drugs in the composition.

As shown in Table 8, the survival rate of T98G cells is dose-dependent on the pharmaceutical composition of the present invention. The active copper ion used in this study was copper gluconate. The experiment results show that when Niclosamide, Disulfiram, and Cu ions were individually administered at concentrations of 8-83 nM, cell survival rates remained relatively high (87.87%-102.19%) with no significant effects. However, when the pharmaceutical composition was administered simultaneously (Niclosamide 4 nM, Disulfiram 8 nM, Cu ions 8 nM), the cell survival rate dropped significantly to 59.99%. By adjusting the concentrations of the three pharmaceuticals, the survival rate of T98G cells could be further reduced to as low as 23.17%. These experiment results confirm that the pharmaceutical composition of the present invention achieves a higher degree of synergistic efficacy.

The experimental results demonstrate that the pharmaceutical composition of the present invention effectively inhibits the survival rate of human glioblastoma cells. The inhibitory effect is positively correlated with the dose adjustments of each pharmaceutical in the composition, leading to a significant reduction in cancer cell survival rates.

### Example 6: Efficacy of the Pharmaceutical Composition of the Present Invention on Human Glioblastoma (U-87) Cell Line

To evaluate the inhibitory effect of the pharmaceutical composition of the present invention on the survival rate of glioblastoma (U-87) cell line, analysis on survival rate was performed. The results are shown in Table 9.

TABLE 9
The Effect of the Pharmaceutical Composition of the Present
Invention on the Survival Rate of Glioblastoma (U-87)
Cell Line. This cancer cell line is used in brain cancer
research and in breast cancer research, too.

| Niclosamide | Disulfiram | Cu | Survival Rate (%) |
|---|---|---|---|
| 8 | — | — | 89.33 |
| 16 | — | — | 88.60 |
| — | 16 | — | 102.66 |
| — | 41 | — | 101.02 |
| — | 83 | — | 63.98 |
| — | — | 16 | 103.71 |
| — | — | 41 | 103.97 |
| — | — | 83 | 101.73 |
| 4 | 8 | 8 | 73.69 |
| 4 | 21 | 21 | 70.48 |
| 4 | 41 | 41 | 30.45 |
| 4 | 83 | 83 | 35.46 |
| 8 | 8 | 8 | 64.63 |
| 8 | 21 | 21 | 62.78 |
| 8 | 41 | 41 | 36.10 |
| 8 | 83 | 83 | 38.99 |
| 16 | 8 | 8 | 58.18 |
| 16 | 21 | 21 | 52.57 |
| 16 | 41 | 41 | 32.93 |
| 16 | 83 | 83 | 35.74 |

The U-87 cell line is known to exhibit high resistance to apoptosis and targeted drugs such as IRESSA (Gefitinib) and ABT-737 (a Bcl-2 inhibitor) (Chang et al., 2011; Cristofanon and Fulda, 2012; Jane et al., 2013).

As shown in Table 9, the experiment results show when Niclosamide, Disulfiram, and pharmacologically active multivalent Cu ions were individually tested, no significant inhibitory effects were observed. However, combinations of various concentration of the pharmaceutical composition of the present invention demonstrated inhibitory effects. When the concentration of Niclosamide is greater than that of Disulfiram and Cu ions, or the concentration of Disulfiram and Cu ions is greater than that of Niclosamide, better inhibitory effects on cancer cells are observed. This suggests a synergistic interaction among the drugs in the composition.

As shown in Table 9, the survival rate of U-87 cells is dose-dependent on the pharmaceutical composition of the present invention. The active copper ion used in this study was copper gluconate. The experiment results shows that when Niclosamide, Disulfiram, and Cu ions were individually administered at concentrations of 8-83 nM, cell survival rates ranged from 63.98% to 103.97%. When Disulfiram was administered alone at 83 nM, the cell survival rate was 63.98%. Using the pharmaceutical composition, the concentrations could be reduced while achieving a higher inhibition rate. When the composition was administered simultaneously (Niclosamide 4 nM, Disulfiram 41 nM, Cu ions 41 nM), the cell survival rate significantly dropped to 30.45%. Compared with using Disulfiram alone, the survival rate of cancer cells is significantly reduced. When the concentrations of the three pharmaceuticals are mutually adjusted, the survival rate of U-87 cancer cells can drop to as low as 30.45%. The experiment results confirm that the pharmaceutical composition of the present invention achieves significantly higher synergistic efficacy on the obviously lower survival rates at specific concentrations of the three pharmaceuticals.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A pharmaceutical composition, comprising:
(a) a targeting agent for a mitochondrion of a cancer cell;
(b) an ion chelating agent; and
(c) a pharmacologically active multivalent ion, wherein the targeting agent for the mitochondrion of the cancer cell is Niclosamide, the ion chelating agent is Disulfiram, and the pharmacologically active multivalent ion is selected from a group consisting of a magnesium ion, a calcium ion, a manganese ion, a ferrous ion, a copper ion, and a zinc ion, and a weight ratio of (a) is 64.5%˜43.7% based on a total weight of the pharmaceutical composition, a weight ratio of (b) is 56.2%˜16.1% based on the total weight of the pharmaceutical composition, and a weight ratio of (c) is 19.4%˜0.000116% based on the total weight of the pharmaceutical composition.

2. The pharmaceutical composition of claim 1, wherein 0.091≤(a)/((b)+(c))≤15.548.

3. The pharmaceutical composition of claim 1, wherein 0.214≤(b)/(c)≤1.8867×10⁴.

4. Use of the pharmaceutical composition as claimed in claim 1 or claim 2 or claim 3 in preparing a medicament for treating a malignant tumor in an individual in need.

5. The use of claim 4, wherein the malignant tumor is selected from a group consisting of a solid tumor and a hematological malignant disease.

6. The use of claim 5, wherein the malignant tumor is selected from a group consisting of lung cancer and brain cancer.

7. The use of claim 4, wherein (a) a dose per administration ranges from 2000 mg to 100 mg, (b) a dose per administration ranges from 500 mg to 128.6 mg, and (c) a dose per administration ranges from 600 mg to 2.65×10⁻⁴ mg.

8. The use of claim 4, wherein administration of (c) is performed no later than administration of (b).

9. The use of claim 4, wherein the medicament is administered no more than twice per day.

10. The use of claim 4, wherein the medicament is administered via an oral route, an injected route, a transdermal route, or an inhaled route.
